(19) United States
(12) Patent Application Publication
(10) Pub. No.: US 2017/0367663 A1
(43) Pub. Date: Dec. 28, 2017

(54) METHOD AND DEVICE FOR EFFECTIVE AUDIBLE ALARM SETTINGS

(71) Applicant: KONINKLIJKE PHILIPS N.V., EINDHOVEN (NL)

(72) Inventors: STIJN DE WAELE, MILLWOOD, NY (US); MUN HUM PARK, EINDHOVEN (NL); ARMIN GERHARD KOHLRAUSCH, EINDHOVEN (NL); ALBERTUS CORNELIS DEN BRINKER, EINDHOVEN (NL); SAM MARTIN JELFS, RIETHOVEN (NL); JOSEPH JAMES FRASSICA, GLOUCESTER, MA (US)

(21) Appl. No.: 15/536,686

(22) PCT Filed: Dec. 11, 2015
(86) PCT No.: PCT/EP2015/079351

§ 371 Date: Jun. 16, 2017

Related U.S. Application Data
(60) Provisional application No. 62/093,489, filed on Dec. 18, 2014.

(30) Foreign Application Priority Data
Jan. 8, 2015 (EP) 15150418.0

(51) Int. Cl. A61B 5/00; G08B 3/10

(52) U.S. Cl. CPC A61B 5/7415; G08B 3/10; A61B 5/0002; A61B 5/746

(57) ABSTRACT

A method for assisting a user in selecting audible alarm settings of a patient monitoring system for monitoring a patient is provided. A synthesis model is generated in response to 1) data indicative of audio features derived from audio recorded in the patient room, and 2) data indicative of vital signs recorded from the patient monitoring system. Sets of audio parameters indicative of respective plurality of audible alarm settings are then processed according to the synthesis model so as to generate respective outputs, e.g. indicative of audibility of the plurality of alarm sounds according to the audible alarm settings synthesized to be played in or outside the patient room. These outputs can then be presented to a user, so as to allow the user to evaluate e.g. alarm audibility and noise level impact of the plurality of audible alarm settings, and e.g. adjust the settings accordingly, such as alarm thresholds etc. The synthesis model may comprise an auralization module, so as to generate synthesized audio outputs to the user. The synthesis model may take into account actual room acoustics in or outside the patient room, so as to allow a precise calculation and/or audio synthesis of an alarm sound to be played in the environment. Alternatively, or additionally, the systhesis model can generate objective and/or subjective metrics that allows the user to find a suitable balance between alarm audibility and impact on noise level.

### FIELD OF THE INVENTION

The present invention relates to the field of methods and devices for audible alarm settings. More specifically, the invention related to effective selection of audible alarm settings for medical equipment, e.g. patient monitoring systems for intensive care.

### BACKGROUND OF THE INVENTION

Noise levels in critical patient care, e.g. in intensive care units at hospitals or medical clinics, are often high due to a combination of noise sources. Some of the most important noise sources are alarm sounds (from patient monitoring systems), staff speech and device noise. High noise levels pose a risk to patients in a medically critical condition, because the noise hampers patient sleep and thus patient recovery. Furthermore, high noise levels increase the probability that caregivers do not perceive alarms that indicate a critical patient condition or, due to alarm fatigue, do not react to the alarm. From the perspective of the clinical staff, the high noise levels increase stress and fatigue, and reduce staff work satisfaction.

Medical audible alarms play an important role in environmental noise. First of all, they constitute a substantial percentage of the total noise power. Moreover, psychological research has shown that patients are more sensitive to alarm sounds compared to other sounds due to their meaning—they could mean that their life is in danger.

Optimized audible alarm settings can strongly reduce the alarm rate, while alarms are still expressed for clinically relevant events, i.e. in the events where vital signs (e.g. blood pressure, heart rate etc.) monitored by the patient monitoring system is selected to trigger an audible alarm. This can significantly reduce the noise caused by the alarm sounds.

However, the users of patient monitoring systems with audible alarms, e.g. clinicians, have no means to evaluate the complete alarm soundscape with new settings compared to the noise level for the currently active settings. To improve alarm audibility the level could be increased, but at the price of a higher general noise level and thus more stressful environment of the patient. There is a lack of quantification of the noise level reduction that is achieved in terms of the noise power. Essentially, this means that clinicians are blind to whether current settings are optimal, and they are blind to the effect of proposed changes in the alarm settings in terms of noise level and audibility of alarms. At best, adjustments of the audible alarm settings are based on the user's gut feeling.

### SUMMARY OF THE INVENTION

In view of the above, it would be advantageous to provide a method and a device for assisting a user, e.g. clinical personnel, in evaluating audible alarms in the selection or adjusting of audible alarm settings of patient monitoring systems with audible alarms for vital sign monitoring. This would allow the user to adjust the audible alarm settings to a preferred balance between alarm detectability and overall noise level in a specific actual critical care environment.

Preferably, the method and device should allow the user to evaluate audibility of an audible alarm setting in an actual environment where a critical patient is located. Preferably, the method and device should allow the user to evaluate the effect of an audible alarm setting on the overall noise level in an actual environment where the critical patient is located.

In a first aspect, the invention provides a method for assisting a user in selecting audible alarm settings of a patient monitoring system for monitoring a patient located in a patient room, the method comprising:
receiving data indicative of audio features derived from audio recorded in the patient room over a period of time, e.g. a period of 24-72 hours,
receiving data indicative of vital signs recorded from the patient monitoring system over a period of time, preferably overlapping with the audio recorded,
optionally, receiving sets of audio parameters indicative of respective plurality of audible alarm settings, e.g. prestored or user input audio parameters,
generating a synthesis model in response to said data indicative of audio features derived from audio recorded in the patient, and in response to said data indicative of vital signs recorded,
processing sets of audio parameters indicative of the respective plurality of audible alarm settings according to said synthesis model, so as to generate respective outputs indicative of alarm sounds according to the plurality of audible alarm settings synthesized to be played in or outside the patient room. Further, the method may comprise presenting said outputs to the user, e.g. so as to allow the user to evaluate audibility of the plurality of audible alarm settings. Further, the method may comprise receiving sets of audio parameters indicative of respective plurality of audible alarm settings, alternatively such data may be prestored.

Such method is advantageous, since it allows the user, e.g. clinical personnel, to evaluate and adjust different audible alarm settings of patient monitoring equipment in a specific acoustic environment, taking into account noise from secondary sources (i.e. sound other than the alarm sound, e.g. speech, foot steps, noise from medical devices etc.) related to specific events taking place simultaneous with an alarm sound. Thus, based on audio features and vital signs recorded in an actual environment, it is possible to generate a synthesis model that can predict audibility metrics, or directly auralize alarm sounds, in response to a synthesis model which is based on actually observed secondary sounds (noise) in case of given vital signs for the actual patient in the actual environment. Thus, such method and systems based thereon, provides insight to the clinician/caregiver in the effects of changing audible alarm settings on the overall noise level and audibility of alarms, since it is possible to synthesize a realistic acoustic environment, or soundscape, in which synthesized alarm sounds can be included. Noise levels may be calculated at a plurality of listener positions, e.g. both positions relevant for a care giver, and the position of the patient so as to evaluate the noise impact on the patient. E.g. the noise levels can be expressed in dB SPL, using the logarithmic decibel scale, or in dB (A), in which the sound spectrum is also weighted according to the spectral sensitivity of human hearing. Still further, the noise level may be expressed as a sound power level of the noise.

Thus, the method provides a valuable tool to test the actual effect of adjustments of audible alarm settings. Audible alarm settings may, apart from audio parameters (e.g. alarm sound level, and melody), comprise one or more parameters indicative of an alarm threshold, e.g. separate threshold settings for different vital parameters. The user can then compare different settings with respect to audibility, i.e. detectability of different audible alarm settings, as well as the effect on the overall noise level. In advanced version of the synthesis model, it is possible to take into account not only average overall secondary sound levels observed in case of an alarm, but also effects in the behavior of humans which depends on the level of an alarm sound. E.g. the effect on speech level which is known as the Lombard effect: speakers increase their speech level in response to a level of an alarm sound and a level of environmental noise.

The synthesized soundscape can be used to derive the audibility of different alarm settings, resulting in effective detection performance statistics (True & False Positive Rate and related metrics) when related to a ground truth for clinical events and by modelling audibility, or by formal listening experiments (auralization). It is to be understood that the plurality of audible alarm settings can be pre-stored settings, or the audible alarm settings can be input or altered by the user, e.g. the user may change the alarm threshold for the Heart Rate High alarm in order to directly compare the audibility and/or noise level effect of different settings. It is to be understood that alarm sound melody and/or level of the alarm sound may be adjusted as well. In versions of the method where e.g. the mentioned Lombard effect and other similar related effects are taken into account, a simple increase in alarm sound level may prove to provide a smaller increase in audibility than expected, due to the related increase in overall noise level in response to the alarm sound. Still further, the method takes into account the room acoustics of the environments, which may further influence the perceived audibility of audible alarms, and also the human response thereto, e.g. higher speech levels in response to a long reverberation time.

The method is suited for implementation on a general computer with a processor programmed to perform the method. Such computer and related program code is a highly suitable tool in alarm management consultancy for hospitals, medical clinics etc. However, it is to be understood that the method may also be implemented as an integrated part of the patient monitoring system itself.

In the following, preferred features and/or embodiments will be described.

The audible alarm settings may, apart from audio parameters, comprise a parameter indicative of an alarm threshold, e.g. one alarm threshold setting for each vital parameter monitored for the patient.

The processing may comprise generating respective outputs indicative of at least one objective metric for each of the plurality of different synthesized audible alarm settings. Especially, such objective metric(s) may comprise a value indicative of a relative level of an alarm sound and a level of secondary sound (noise) estimated to be present simultaneously with said alarm sound. This may allow a measure of audibility of an alarm sound in a setting to be calculated in response to the synthesis model which is easy to communicate to the user, since it can be expressed and displayed to the user as one single number. Preferably said level of the secondary sound is determined in accordance with the audio features derived from the recorded audio and the vital sign data, thus indicating actually measured noise levels in the specific setup.

The processing may comprise processing according to an algorithm involving a psycho-acoustic model, so as to generate respective output values indicative of audibility of each of the plurality of different synthesized audible alarm settings. Thus in such embodiments the audibility can be directly calculated, including e.g. psychoacoustic masking effects such as spectral and temporal masking effects, thus allowing a user to receive an output where audibility of the alarm sound of a given audible alarm setting, e.g. expressed as one single value which can be displayed to the user. If preferred, overall noise level can further be calculated and output to the user, so as to allow the user to evaluate a balance between alarm audibility and overall noise level. Especially, said output values indicative of audibility of each of the plurality of different synthesized audible alarm settings may comprise values indicative of calculated alarm detection performance, such as estimated statistics of True & False Positive detection Rate and related metrics.

The method may comprise an auralization algorithm arranged to generate respective audio signals according to said plurality of audible alarm settings, and further comprising presenting said plurality of audio signals to the user. Such auralization allows the user to actually listen to a synthesized or virtual reality version of the audible alarm settings synthesized to be played in the actual acoustic environments including, preferably, secondary sounds (noise) as well as the alarm sound. This allows the user to evaluate audibility of the alarm sound and overall noise level in the actual environment without the need to actually play the alarm sound in clinical setting. Thereby, the user can adjust the audible alarm setting to obtain an optimal balance between alarm audibility (detection) and overall noise level also taking into account subjective preferences, e.g. with respect to alarm sound melody etc. Especially, said auralization algorithm may comprise generating said respective audio signals for a synthesized listener position in or outside the patient room, thereby allowing the user to evaluate an alarm sound at different positions, e.g. in the patient room, in a monitoring room, or in a corridor etc. taking into account both the different room acoustics and secondary sounds in the different locations.

Especially, the method may comprise convolving characteristics of an alarm sound in an alarm setting with an impulse response based on a measure of electroacoustic impulse response for an electroacoustic transducer intended to play audible alarm sounds, corresponding to a listener position in or outside the patient room, so as to generate said plurality of audio signals to the user synthesizing the user being present at the listener position. This allows a realistic synthesis model which takes into account the actual acoustic transfer function from electroacoustic transducer (loudspeaker) position to a target listener position. This is relevant for an accurate modelling for calculation of precise objective and subjective audibility metrics as well as in version comprising an auralization algorithm. Especially, the method may comprise convolving a simulated or measured impulse response from the electroacoustic transducer to a listener position with a synthesized alarm sound, so as to generate a resulting synthesized alarm sound at the listener position. This may comprise modelling the acoustic environment around the electroacoustic transducer and the listener position.

The method may comprise processing according to an algorithm arranged to generate a value indicative of an estimated noise level in or outside the patient room for each of the plurality of audible alarm settings synthesized to be played. Together with an objective and/or subjective metric indicative of audible alarm audibility, the user has the possibility to evaluate a balance between audibility, i.e. detection of the audible alarm, and the consequence on the overall noise level.

The method may comprise receiving data indicative of room acoustics in or outside the patient room, and wherein the synthesis model is generated in accordance with said data indicative of room acoustics in or outside the patient room. To take into account the actual room acoustics, e.g. directly measured or modelled based on geometric inputs from the actual environment, it is possible to more precisely calculate alarm audibility and noise level and/or provide a more accurate auralization.

The method may further comprise receiving alarm data indicative of at least audible alarm types and time stamps of audible alarms of the patient monitoring system corresponding to at least part of said period of time from which the audio features are derived. Such data allows correlation between audio features in the recorded audio and the recorded vital signs, e.g. so as to automatically detect differences between alarm sound and secondary sounds in the recorded audio, e.g. to allow a better estimate of secondary sounds from different events occurring simultaneous with an alarm. Especially, it may be preferred to store sets of detected sound levels for a plurality of different identified events happening as a consequence of each of a plurality of different audible alarm types. E.g. events such as speech, sound from a pager, sound from an intubation event, etc.

The method may comprise synthesizing speech sound by spectrally and temporally shaped noise, thus providing a synthesized sound matching the psycho-acoustic masking effect of speech, but avoiding the information content of recorded speech. Hereby a precise alarm audibility synthesis model can be generated without the need to use recorded speech.

The method may comprise adding synthesized audible alarm sound to secondary sound. Especially, said secondary sound may be generated in response to a correlation between alarm events and secondary sound based on said audio features derived from audio recorded in the patient room over a period of time. Said secondary sound may comprise actually recorded sound, e.g. sound from specific events identified in the recorded audio. Especially, said secondary sound may comprise synthesized speech, and especially a level of said synthesized speech is adjusted in response to a level of a synthesized audible alarm sound, hereby including the so-called Lombard effect. Especially, the method may comprise analyzing the audio features so as to determine a level of secondary sound in response to a plurality of different events for each of the plurality of audible alarm settings.

It may be preferred to derive said audio features in real time during recording of audio in the patient room, thus avoiding storing the directly recorded sound which may include private speech etc.

Said audio parameters for an audible alarm setting comprise data indicative of one or more of: alarm condition configuration, alarm reminder setting, alarm sound melody, alarm sound level, alarm sound duration, and alarm severity.

Said audio features derived from recorded audio may comprise: short-time spectral representations, level distributions, and long-term spectra.

It is to b understood that the mentioned embodiments can be combined. E.g. the method may comprise more than one output for each audible alarm setting. E.g. the method may comprise for each audible alarm setting: an auralization output as well as one or more values indicative of objective metrics related to audibility and/or one or more direct calculations of audibility and/or an output indicative of a noise level.

In a second aspect, the invention provides a computer executable program code adapted to perform the method according to the method according to the first aspect, when executed on a processor. Such computer executable program code is thus capable of performing the steps of the method according to the first aspect which can be implemented in software, e.g. as an add-on or modification of existing software in an alarm management consulting system. E.g. such software code is suited for execution on a tablet, a laptop, a general computer, or a dedicated device. The computer executable program code may especially be present on a non-transitory computer readable storage medium, or it may be loaded into memory of a processor system arranged to execute the program code. Especially, the computer executable program code may be adapted for a processor forming part of a patient monitoring system.

In a third aspect, the invention provides a device or a system arranged to assist a user in selecting audible alarm settings of a patient monitoring system for monitoring a patient located in a patient room, the device comprising
a processor programmed:
to receive data indicative of audio features derived from audio recorded in the patient room over a period of time,
to receiving data indicative of vital signs recorded from the patient monitoring system over a period of time,
optionally, to receive sets of audio parameters indicative of respective plurality of audible alarm settings, and
to generate a synthesis model in response to said data indicative of audio features derived from audio recorded in the patient room over a period of time, and said data indicative of vital signs recorded over a period of time,
to process sets of audio parameters indicative of the respective plurality of audible alarm settings according to said synthesis model, so as to generate respective outputs indicative of alarm sound according to the plurality of audible alarm settings synthesized to be played in or outside the patient room.

The device or system may further comprise a user interface arranged to present said outputs to a user, e.g. so as to allow the user to evaluate audibility of the plurality of audible alarm settings.

Especially, the device may be an assisting tool to assist a user or a consultant in alarm management of an intensive care unit at a hospital, medical clinic, or the like. Preferably, the device comprises a user interface comprising a display to present the user with the outputs in the form of values indicative of the calculated objective and/or subjective metrics, or values directly indicative of audibility of the plurality of audible alarm settings.

The device may comprise an audio output interface comprising a loudspeaker and/or headphone to present the result of an auralization of the plurality of audible alarm settings to the user. Such audio output interface may present an audio output to the user as one of: a one channel output, a stereo output, and a 3D audio output, e.g. using a surround sound setup, or using binaural technique such as based on measured or synthesized head-related transfer functions.

The device may comprise an audio input interface with a microphone arranged for position in the patient room, and wherein the device is arranged to record audio in the patient room, preferably over a period of up to 12, 24, 48 or 64 hours or even more. The device may be arranged to derive audio features from audio recorded in the patient room over a period of time, and thus provide these audio features to the above described processing. The device may allow a user to manually annotate the recording so as to identify sound/noise sources in the recording, e.g. such manual annotation may be part of the deriving of said audio features. Preferably, the device further comprises an input arranged to record vital signs data from the patient monitoring system simultaneous with the audio recording, so as to allow identification of audio in the patient room during clinically (and possibly alarm) relevant events.

In a special embodiment, the device according to the third aspect forms part of a patient monitoring system comprising a programmable audible alarm system. In such embodiment, the patient monitoring system itself assists the user in obtaining an optimal audible alarm setting in the actual environment by providing the user with guiding towards obtaining a balance between audibility of the various alarms and the overall noise level in or outside the patient room.

It is appreciated that the same advantages and embodiments of the first aspect apply as well for the second and third aspects. In general the first, second and third aspects may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 illustrates steps of a method embodiment,

FIG. 2 illustrates a block diagram of a device embodiment,

FIG. 3 illustrates an embodiment comprising auralization, and

FIG. 4 illustrates an example of implementation of soundscape synthesis based on a given generated alarm.

### DESCRIPTION OF EMBODIMENTS

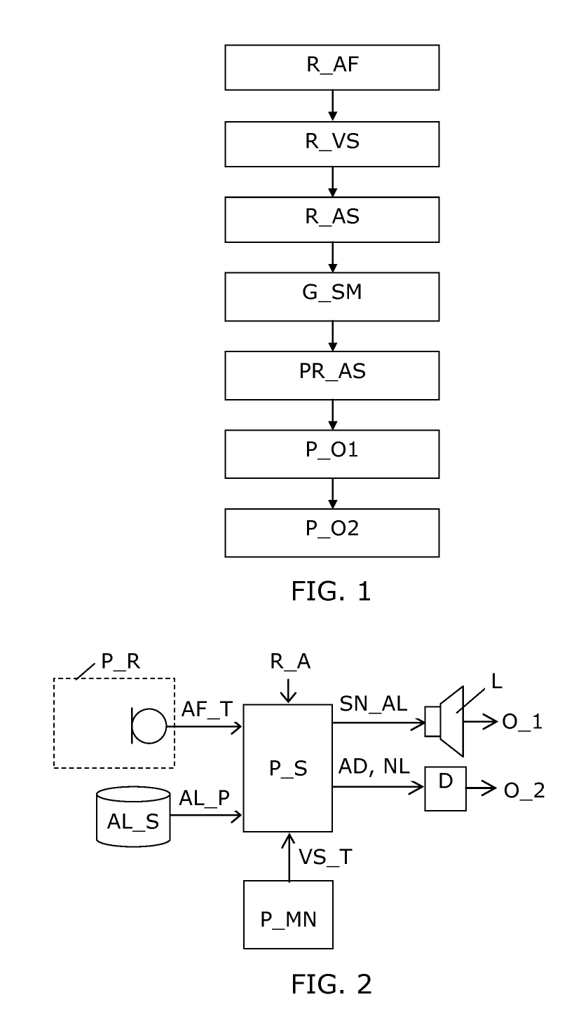

FIG. 1 shows an embodiment of a method for assisting a user in selecting audible alarm settings of a patient monitoring system for monitoring a patient located in a patient room. The method comprises receiving of inputs, namely: data indicative of audio features R_AF derived from audio recorded in the patient room over a period of time, data indicative of vital signs R_VS recorded from the patient monitoring system over a period of time, and sets of audio parameters indicative of respective plurality of audible alarm settings R_AS.

A synthesis model is the generated G_SM in response to the audio features and vital signs. This synthesis model preferably takes into account different sound sources identified in the audio features, including precise levels of noise and spectral content of the noise generated for each of the various sources, in order to provide a synthesis model with information about background noise and preferably also description of room acoustics of the relevant room(s) where the audible alarm sound is intended to be played. Thus, the synthesis model is preferably capable of taking as input audio parameters for each audible alarm settings, and thus processing the sets of audio parameters indicative of the respective plurality of audible alarm settings PR_AS accordingly. The audio features and vital signs recorded are combined to identify sound sources, or sound events, occurring in connection with vital signs triggering an alarm according to a given audible alarm setting (e.g. including alarm trigger level settings for each vital sign monitored). In other words, the synthesis model is capable of generating a synthesized sound landscape or soundscape of an alarm sound of an audible alarm setting being played in a given sound environment.

As output from the synthesis model, respective outputs are generated indicative of audibility of the plurality of audible alarm settings synthesized to be played in or outside the patient room, preferably taking into account noise (secondary sound sources) derived from the audio features from the recorded audio, and also taking into account the vital signs from the patient recorded, e.g. comprising heart rate, blood pressure data etc. By combining these data, a realistic soundscape can be generated taking into account secondary sound (noise) related to specific events, e.g. speech or machine sounds related to specific types of alarms, or merely taking into account an average background noise level.

The synthesis model may comprise an auralization algorithm which results in an audio signal which allows the user to hear a synthesized version of an audible alarm sound being played, preferably including correct level, melody, durating and other characteristics of the alarm sound of an audible alarm setting, in relation to background noise from various secondary sound sources, thus allowing the user to subjectively evaluate audibility of the given alarm setting. In addition, or instead, the synthesis model may comprise an algorithm arranged to calculate an objective metric, e.g. level of alarm sound in dB relative to a level of secondary sounds (noise) typically present simultaneously with the alarm sound. In addition, or instead, the synthesis model may comprise an algorithm arranged to directly calculate a measure of audibility by psycho-acoustic model taking into account e.g. masking effects.

In the illustrated embodiment an audio output is generated as a result of an auralization as a first output, and an objective audibility measure of alarm sound level relative to secondary sound (noise) level is generated as a second output. Finally, the outputs are presented to the user, so as to allow the user to evaluate audibility of the plurality of audible alarm settings. In the illustrated P_O1, P_O2 embodiment comprising presenting P_O1 the audio signal to the user via a headphone or loudspeaker(s), and presenting P_O2 the displaying a value indicative of the objective measure of alarm sound level relative to a secondary sound (noise) level.

It is to be understood that the method is suitable for execution on a computer programmed to implement the method. E.g. such computer can form part of an alarm management system intended for consultancy regarding alarm management at a hospital or a medical clinic, and thus the method can be used to advise a hospital to improve their alarm management and reduce noise in their wards, still with due respect to ensure that audibility of the audible alarms are adapted to the actual acoustical surroundings. The method may be implemented as software to be executed on a laptop computer etc. Outputs to the user can be generated by the laptop display and/or auralization sound via sound card or USB output.

FIG. 2 shows a block diagram of a device embodiment where audio is recorded by a microphone in a patient room P_R where an intensive care patient is located. Audio features versus time AF_T are derived from the audio recorded, and applied to a processor system P_S. Different audible alarm setting data AL_S are stored and applied to the processor system P_S, or applied to the processor system P_S via a user interface by a user. Vital signs versus time VS_T are also applied to the processor system P_S in the form of output from a patient monitoring system P_MN, e.g. heart rate, blood pressure and other patient relevant parameters which may be monitored. In addition, data indicative of room acoustics R_A of the patient room P_R and e.g. other related locations where alarm sound is intended to be played, is also applied to the processor system P_S.

The processor system P_S, e.g. a tablet, a laptop computer etc. is programmed according to the just described method, thus causing the processor system P_S to generate two outputs O_1 and O_2. Namely O_1: an auralization output in the form of synthesized alarm sound SN_AL which is presented to the user via an electroacoustic transducer, e.g. a loudspeaker L, arranged to generate auralized sound to the user taking into account audio parameters for the audible alarm settings AL_P, secondary sound sources (noise) modelled in response to recorded audio features AF_T and vital signs VS_T, as well as the room acoustics, so as to arrive at a realistic synthesized soundscape. Further, a second output O_2 comprises values indicative of alarm audibility AD, as well as a general noise level NL resulting from the given audible alarm setting AL_S.

The result is that the user is provided with a valuable tool to evaluate various parameters that may be adjusted for audible alarm settings, since audibility of the alarm sounds as well as the result on the overall noise level in the environment can be evaluated—subjectively as well as based on objective parameters.

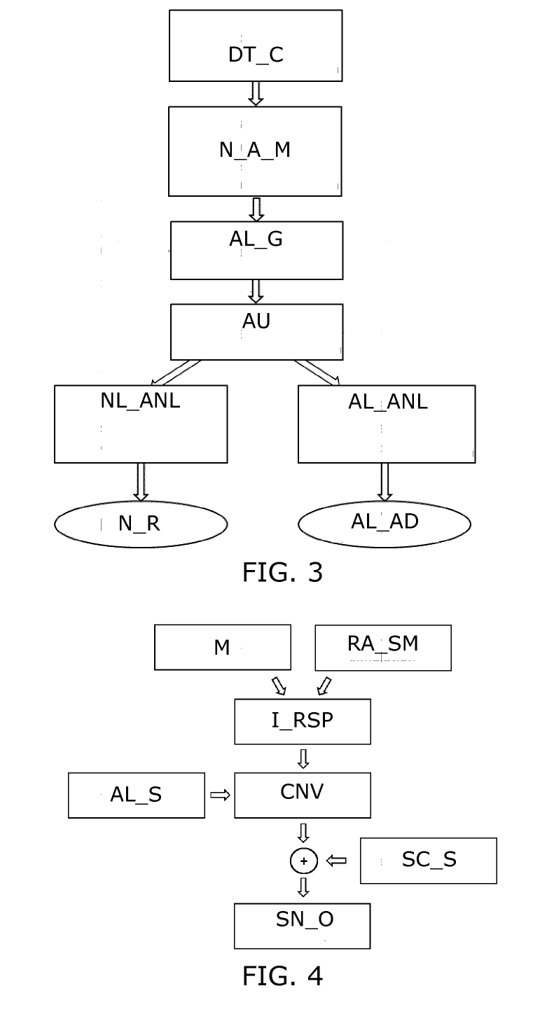

FIG. 3 shows a flow chart for an embodiment comprising auralization. Reference is made to the general method already described in connection with FIG. 1. Details regarding the various steps will be given below. A first step comprises data collection DT_C which involves collecting audio features, vital signs, as well as parameters indicative of various audible alarm settings. A synthesis model involving noise and audible alarm modelling N_A_M serves to combine the collected data to arrive at a model of alarm sound, e.g. taking into account room acoustics, as well as secondary sound (noise related to the alarms). E.g. a set of (average) noise levels for related alarm types and activities or events may be generated in response to the collected data and stored. E.g. activities or events may comprise such as: speech, noise from a pager, noise from intubation, ventilator noise, plastic unripping, drawer closing, computer keyboard typing, water splashing etc. An average noise level for a number of observed events can then be stored for each of a plurality of alarm types. Alarm generation AL_G can then be performed by processing parameters for a given audible alarm setting in the synthesis model, e.g. involving sound level and melody etc. for a given alarm type, and taking into account noise from various events related thereto. The alarm generation AL_G may receive as input tunable parameters, such as re-alarm being on or off, and a configuration of alarm condition. By ‘re-alarm’ is understood: as long as the alarm condition is still met, the alarm is raised again after a specified amount of time (e.g. 1 minute) after the alarm has been silenced. Given parameters may comprise such as: average silencing time, frequency of InOps etc. regarding the generated alarms.

In this embodiment, the output from the alarm generation AL_G is applied to an auralization module AU which generates an audio signal accordingly. Tunable or at least partly tunable parameters comprise: alarm volume (level) and melody, a listener position, and alarm rendering location (position of loudspeakers generating the alarm sound). Given parameters comprise: room acoustics, sound caused by alarm-related caregiver actions, and environmental noise. The generated audio signal is applied to a noise level analyzing algorithm NL_ANL which calculates subjective and objective noise level measures for both patient and caregiver locations, and outputs a noise report N_R accordingly. Further, an alarm audibility analysis algorithm AL_ANL calculates subjective and objective measures of audibility of the given alarm sound according to an audible alarm setting, and an alarm audibility AL_AD performance report is output accordingly.

Regarding the collection of data, examples are given in the following. The data for a given audio alarm setting may comprise: time stamps, alarm type, alarm duration, and caregiver acknowledgement (e.g. on/off). The vital signs may comprise: heart rate, blood pressure etc., at a time resolution that is sufficiently accurate for efficient alarm regeneration, e.g. typically a 1 second sampling interval is sufficient. The audio features may be derived from audio recorded by microphones placed in the patient room(s). To prevent privacy issues of recorded data, raw audio is preferably not stored. Instead, a modelling stage may be performed in real time to derive audio features which are then stored, preferably together with time information.

The synthesis model, and the alarm sound generation therein, may take into account one or more, or all of:
1. Patient monitor settings, alarm condition configuration (limits, on/off alarm setting, yellow/short yellow setting, . . . ), alarm reminder (activated or not—if active, an audible alarm is started again after a certain period of time since the alarm was acknowledged, if the alarm condition is still active), alarm volume (i.e. level) and alarm melody. E.g. it may be taken into account that sound level and profile can be set per alarm severity. The short (yellow) alarm only sounds for 5 seconds, while other alarms sound until acknowledged.
2. Alarm severity (Red (severe), Yellow (not severe), Short Yellow (even less severe), InOp (i.e. alarm if one or more monitored vital signs fail to operate or fail to be reliable))
3. Room acoustics of the acoustic environment where the alarm sound is intended to be played.
4.Listener location, e.g. in patient bed, bedside, nurse station, or other.
5. Alarm rendering location(s), e.g. patient monitor or nurse station.
6. Time (e.g. average time) till alarm silencing.
7. Frequency of InOps (i.e. alarm if one or more monitored vital signs fail to operate or fail to be reliable) independent of vital signs.
8. Secondary sounds generated in response to the alarm, i.e. speech, step sounds, machine sounds related to various events.

The mentioned variables 1-6 can be determined either directly or with existing algorithms by combining the alarms/vitals recording and audio recording. For secondary sounds caused in response to an alarm (8), it is proposed to record a short time interval of audio triggered by the alarm. By aggregating the alarm sound (or derived features) of these responses per alarm type and per noise source (activity), a model is obtained for the secondary sounds.

For speech, the model includes speech that occurs in response to an alarm. A further phenomenon that is modelled is the Lombard effect: Speakers increase their speech volume in response to the general level of alarm and environmental noise.

A characterization of the environmental noise is obtained by analysis of the other sounds not caused by audible alarms. It is noted that these sounds need to be adapted as well since presumably the alarm sounds have effects on the behaviors of staff and patients. E.g. with a lower alarm rate, one may assume less alarm fatigue with the staff, which affects their response to alarms. For example, quicker responses will reduce the occurrence of alarm reminders. Such effects can be built into the model as well.

Alarm regeneration is performed with new alarm settings that are of interest to the clinical site. The frequency of InOps is used to generate a realistic level of InOp alarms. This component has the following outputs: generated alarm a) start, b) duration, c) severity (Red (severe), Yellow (less severe)), and d) modality (heart rate, respiration rate, . . . ).

The characteristics of the alarms sounds may be altered according to the given audible alarm settings. Impulse responses from the transducers that generate alarm sounds (which are typically built into life-supporting or monitoring devices) to the possible listening positions, for example, at the patient bed, at the bedside (at standing level), in the hallway, or at the nursing station may be pre-measured or simulated. These impulse responses can then be convolved with the generated alarms (see above), and finally, a realistic soundscape at the receiver (listening) position(s) can be synthesized by adding the secondary sounds.

The secondary sound is preferably generated by exploiting the observed correlation between alarm events and secondary sounds. Notably, a synthesized speech level may be adjusted according to the generated alarm sound level. For example, if the new settings generate fewer alarms, the speech level may be decreased accordingly. A challenge with synthesis of speech as a secondary sound is that the speech recorded during the data collection cannot be auralized as is due to privacy concerns. Therefore, white noise shaped in the temporal and spectral domain to be similar to speech is used instead. The benefit of using this replacement signal is that it still allows for an accurate evaluation of the audibility of the alarms, since alarm masking properties of the synthesized speech-like signal are very similar to those of actual speech.

An auralized sound sample can be generated to reflect several conditions. For example, a sound sample can be generated that reflects the average soundscape, by generating a sample where the generated alarm rate is equal to the average alarm rate; and the secondary sound levels are equal to average levels per sound category. Similarly, a sound sample can be generated for a time of peak alarm load, or for a night time period.

FIG. 4 illustrates one possible way of generating a synthesized soundscape output SN_O in a synthesis model embodiment. A measured M or room acoustic simulated RA_SM impulse response I_RSP indicative of sound transmission from an electroacoustic transducer (loudspeaker) playing a generated alarm sound AL_S to a listener position, e.g. a position of the patient, or a location of a caregiver. This impulse response is convolved CNV with the generated alarm sound and subsequently added to secondary sounds SC_S, thus resulting in a total synthesized soundscape output SN_O.

The synthesized secondary sound (i.e. noise) can be analyzed objectively by calculating the noise energy (e.g. expressed in dB). The noise can be evaluated subjectively by rendering the sounds to listeners. The noise can be rendered alternating for different alarm settings to enable the subjective comparison. The sound can be evaluated by a panel of listeners, who rate the difference between 2 settings using a calibrated scale, e.g. a five-point Likert scale, with possible response entries: Setting a) is: much less noisy; less noisy; equally noisy; more noisy; much more noise than setting b). Also, the noises can be rendered for the customer to illustrate the results of the calculated noise energy with actual audio.

The audibility of alarms may be analyzed objectively by application of perception models to the generated sound. For example, an alarm sound may be masked by other sounds (alarm or background), making it not observable by a listener. Subjective evaluation may be performed by asking a clinical user to listen to the rendered audio and to identify alarms that occur in the audio. Some alarms are ignored because they are not audible. These alarms may not be counted as a true positive in the calculation of the effective true positive rate for the audible alarms. Actions that may positively impact the effective true positive rate are: 1) reduction of the overall alarm rate by widening alarm limits, 2) reduction of the environmental noise, 3) adjustment of the alarm level and melody, and 4) strategic placement of speakers rendering the audio.

In some embodiments, estimation of sound level and alarm audibility is calculated directly. This may comprise a sound synthesis step being replaced by a simplified soundscape where the levels for individual sound events are derived from averaged sound levels (population average). In this step, the sound energy per category, e.g. ‘alarm’ and ‘other’ is calculated directly, instead of using auralization as an intermediate step. Possibly, sound levels can be further investigated in different frequency bands, to enhance the subsequent calculation of audibility. This embodiment requires less precise audio data to be available. Also, an initial estimate can be done based on simpler models of acoustic propagation.

To sum up, the invention provides a method for assisting a user in selecting audible alarm settings of a patient monitoring system for monitoring a patient. A synthesis model is generated in response to 1) data indicative of audio features derived from audio recorded in the patient room, and 2) data indicative of vital signs recorded from the patient monitoring system. Sets of audio parameters indicative of respective plurality of audible alarm settings are then processed according to the synthesis model so as to generate respective outputs, e.g. indicative of audibility of the plurality of alarm sounds according to the audible alarm settings synthesized to be played in or outside the patient room. These outputs can then be presented to a user, so as to allow the user to evaluate e.g. alarm audibility and noise level impact of the plurality of audible alarm settings, and e.g. adjust the settings accordingly, such as alarm thresholds etc. The synthesis model may comprise an auralization module, so as to generate synthesized audio outputs to the user. The synthesis model may take into account actual room acoustics in or outside the patient room, so as to allow a precise calculation and/or audio synthesis of an alarm sound to be played in the environment. Alternatively, or additionally, the synthesis model can generate objective and/or subjective metrics that allows the user to find a suitable balance between alarm audibility and impact on noise level.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. Method for assisting a user in selecting audible alarm settings of a patient monitoring system for monitoring a patient located in a patient room, the method comprising:
receiving (R_AF) data indicative of audio features derived from audio recorded in the patient room over a period of time,
receiving (R_VS) data indicative of vital signs recorded from the patient monitoring system over a period of time,
generating (G_SM) a synthesis model in response to said data indicative of audio features derived from audio recorded in the patient, and in response to said data indicative of vital signs recorded, and
processing (PR_AS) sets of audio parameters indicative of respective plurality of audible alarm settings according to said synthesis model, so as to generate respective outputs indicative of alarm sounds according to the plurality of audible alarm settings synthesized to be played in or outside the patient room.

2. Method according to claim 1, wherein said processing (PR_AS) comprises generating respective outputs (P_O1, P_O2) indicative of at least one objective metric for each of the plurality of different synthesized audible alarm settings.

3. Method according to claim 1, wherein said at least one objective metric comprises a value indicative of a relative level of an alarm sound and a level of secondary sound estimated to be present concurrent with said alarm sound.

4. Method according to claim 1, comprising processing according to an algorithm involving a psycho-acoustic model, so as to generate respective output values indicative of audibility of each of the plurality of different synthesized audible alarm settings.

5. Method according to claim 4, wherein said output values indicative of audibility of each of the plurality of different synthesized audible alarm settings comprises values indicative of calculated alarm detection performance.

6. Method according to claim 1, comprising an auralization algorithm arranged to generate respective audio signals according to said plurality of audible alarm settings, and further comprising presenting said plurality of audio signals (O_1) to the user.

7. Method according to claim 6, wherein said auralization algorithm comprises generating said respective audio signals for a synthesized listener position in or outside the patient room.

8. Method according to claim 1, wherein said respective outputs are indicative of audibility of audible alarm sounds according to the plurality of audible alarm settings synthesized to be played in or outside the patient room.

9. Method according to claim 1, comprising convolving characteristics of an alarm sound in an alarm setting with an impulse response based on a measure of electroacoustic impulse response for an electroacoustic transducer intended to play audible alarm sounds, corresponding to a listener position in or outside the patient room, so as to generate said plurality of audio signals to the user synthesizing the user being present at the listener position.

10. Method according to claim 1, comprising processing according to an algorithm arranged to generate a value indicative of an estimated noise level (NL) in or outside the patient room for each of the plurality of audible alarm settings synthesized to be played.

11. Method according to claim 1, comprising receiving data indicative of room acoustics in or outside the patient room, and wherein the synthesis model is generated (G_SM) in accordance with said data indicative of room acoustics in or outside the patient room.

12. Method according to claim 1, wherein the set of audible alarm settings comprises a parameter indicative of an alarm threshold.

13. Method according to claim 1, comprising adding synthesized audible alarm sound (AL_S) to secondary sound (SC_S), and wherein said secondary sound (SC_S) is generated in response to a correlation between alarm events and secondary sound based on said audio features derived from audio recorded in the patient room over a period of time.

14. A computer executable program code adapted to perform the method according to the method according to claim 1, when executed on a processor (P_S).

15. A device arranged to assist a user in selecting audible alarm settings of a patient monitoring system (P_MN) for monitoring a patient located in a patient room, the device comprising a processor (P_S) programmed:
to receive data indicative of audio features (AF_T) derived from audio recorded in the patient room (P_R) over a period of time,
to receiving data indicative of vital signs (VS_T) recorded from the patient monitoring system (P_MN) over a period of time,
to generate a synthesis model in response to said data indicative of audio features (AF_T) derived from audio recorded in the patient room (P_R) over a period of time, and said data indicative of vital signs (VS_T) recorded, and
to process sets of audio parameters (AL_P) indicative of respective plurality of audible alarm settings according to said synthesis model, so as to generate respective outputs (SN_AL, AD, NL) indicative of alarm sounds according to the plurality of audible alarm settings synthesized to be played in or outside the patient room (P_R).
